(19) United States
(12) Patent Application Publication
(10) Pub. No.: US 2025/0107730 A1
(43) Pub. Date: Apr. 3, 2025

(54) TOTAL HEMOGLOBIN SCREENING SENSOR

(71) Applicant: Masimo Corporation, Irvine, CA (US)

(72) Inventor: Ammar Al-Ali, San Juan Capistrano, CA (US)

(21) Appl. No.: 18/979,259

(22) Filed: Dec. 12, 2024

Related U.S. Application Data
(63) Continuation of application No. 17/550,189, filed on Dec. 14, 2021, now Pat. No. 12,201,420.
Application No. 17/550,189 is a continuation of application No. 16/268,345, filed on Feb. 5, 2019, now Pat. No. 11,331,013.
Application No. 16/268,345 is a continuation of application No. 14/845,090, filed on Sep. 3, 2015, now Pat. No. 10,231,657.
(60) Provisional application No. 62/045,565, filed on Sep. 4, 2014.

(51) Int. Cl. A61B 5/1455; A61B 5/00; A61B 5/0205; G16H 40/63; G16H 50/30

(52) U.S. Cl. CPC A61B 5/1455; A61B 5/14552; A61B 5/6826; A61B 5/7221; A61B 5/7275; G16H 40/63; G16H 50/30; A61B 5/02055; A61B 5/742; A61B 5/746; A61B 2560/0443

(57) ABSTRACT

A total hemoglobin index system derives total hemoglobin (tHb) index value utilizing a depopulated emitter with an index set of LEDs. A patient fails a tHb index test if tHb measurements are trending down and/or tHb values fall below a predefined index threshold. If a patient fails the tHb index test, a high resolution sensor derives a specific tHb measurement utilizing a high resolution set of LEDs. The number of high resolution set LEDs is greater than the number of index set LEDs.

### INCORPORATION BY REFERENCE TO ANY PRIORITY APPLICATIONS

The present application is a continuation of U.S. patent application Ser. No. 17/550,189, filed Dec. 14, 2021, titled “TOTAL HEMOGLOBIN SCREENING SENSOR,” which is a continuation of U.S. patent application Ser. No. 16/268,345, filed Feb. 5, 2019, titled “TOTAL HEMOGLOBIN SCREENING SENSOR,” now U.S. Pat. No. 11,331,013, which is a continuation of U.S. patent application Ser. No. 14/845,090, filed Sep. 3, 2015, titled “TOTAL HEMOGLOBIN SCREENING SENSOR,” now U.S. Pat. No. 10,231,657, which claims priority benefit under 35 U.S.C. § 119(e) to U.S. Provisional Patent Application Ser. No. 62/045,565 filed Sep. 4, 2014, titled Total Hemoglobin Screening Sensor, hereby incorporated in its entirety by reference herein.

Any and all applications for which a foreign or domestic priority claim is identified in the Application Data Sheet as filed with the present application are hereby incorporated by reference under 37 CFR 1.57.

### BACKGROUND OF THE INVENTION

Pulse oximetry systems for measuring constituents of circulating blood have gained rapid acceptance in a wide variety of medical applications, including surgical wards, intensive care and neonatal units, general wards, home care, physical training, and virtually all types of monitoring scenarios. A pulse oximetry system generally includes an optical sensor applied to a patient, a monitor for processing sensor signals and displaying results and a patient cable electrically interconnecting the sensor and the monitor. A pulse oximetry sensor has light emitting diodes (LEDs), typically one emitting a red wavelength and one emitting an infrared (IR) wavelength, and a photodiode detector. The emitters and detector are attached to a patient tissue site, such as a finger. The patient cable transmits drive signals to these emitters from the monitor, and the emitters respond to the drive signals to transmit light into the tissue site. The detector generates a signal responsive to the emitted light after attenuation by pulsatile blood flow within the tissue site. The patient cable transmits the detector signal to the monitor, which processes the signal to provide a numerical readout of physiological parameters such as oxygen saturation (SpO₂) and pulse rate. Advanced physiological monitoring systems utilize multiple wavelength sensors and multiple parameter monitors to provide enhanced measurement capabilities including, for example, the measurement of carboxyhemoglobin (HbCO), methemoglobin (HbMet) and total hemoglobin (tHb).

Pulse oximeters capable of reading through motion induced noise are disclosed in at least U.S. Pat. Nos. 6,770,028, 6,658,276, 6,650,917, 6,157,850, 6,002,952, 5,769,785, and 5,758,644; low noise pulse oximetry sensors are disclosed in at least U.S. Pat. Nos. 6,088,607 and 5,782,757; all of which are assigned to Masimo Corporation, Irvine, California (“Masimo”) and are incorporated in their entireties by reference herein.

Physiological monitors and corresponding multiple wavelength optical sensors are described in at least U.S. patent application Ser. No. 11/367,013, filed Mar. 1, 2006 and entitled Multiple Wavelength Sensor Emitters and U.S. patent application Ser. No. 11/366,208, filed Mar. 1, 2006 and entitled Noninvasive Multi-Parameter Patient Monitor, both assigned to Cercacor Laboratories, Irvine, CA (Cercacor) and both incorporated in their entireties by reference herein.

Further, physiological monitoring systems that include low noise optical sensors and pulse oximetry monitors, such as any of LNOP® adhesive or reusable sensors, SofTouch™ sensors, Hi-Fi Trauma™ or Blue™ sensors; and any of Radical®, SatShare™, Rad-9™, Rad-5™, Rad-5v™ or PPO+™ Masimo SET® pulse oximeters, are all available from Masimo. Physiological monitoring systems including multiple wavelength sensors and corresponding noninvasive blood parameter monitors, such as Rainbow™ adhesive and reusable sensors and RAD-57™ and Radical-7™ monitors for measuring SpO₂, pulse rate, perfusion index, signal quality, HbCO and HbMet among other parameters are also available from Masimo.

### SUMMARY OF THE INVENTION

Occult bleeding is frequent in surgery, intensive care and obstetrics, and late detection increases the corresponding risks of serious injury or death. Bleeding alone is responsible for 19% of in-hospital maternal deaths. Further, bleeding significantly increases the total cost of patient treatment. Total hemoglobin (tHb) measurements identify almost 90% of patients with bleeding, but traditional lab measurements are infrequent and delayed. Advantageously, a tHb index system is an aid to clinicians in intensive care units and labor and delivery wards to detect occult bleeding.

Traditional invasive lab testing provides delayed results and requires a painful needle stick and time-consuming blood draws. A total hemoglobin (tHb) index system incorporating an advantageous noninvasive, disposable sensor and a monitor advantageously calculating and displaying a tHb index facilitates timely patient assessment and reduces the need to wait for lab results.

Also, noninvasive tHb monitoring advantageously provides real-time visibility to changes, or lack of changes, in total hemoglobin between invasive blood sampling. Continuous, real-time tHb monitoring is particularly advantageous when a tHb trend is stable and a clinician may otherwise think tHb is dropping; a tHb trend is rising and the clinician may otherwise think tHb is not rising fast enough; or the tHb trend is dropping and the clinician may otherwise think tHb is stable.

Further, a tHb index system decreases the risk of accidental needle sticks and exposure to blood-borne pathogens. In addition, a disposable tHb sensor requires no lab consumables or waste disposal, reduces painful needle sticks and time-consuming blood draws and enables immediate face-to-face counseling with a clinician. The advantages of a tHb index system are enhanced through the use of a low-cost tHb index sensor embodiment utilizing a reduced number of LEDs in the emitter, as described below.

### BRIEF DESCRIPTION OF THE DRAWINGS

FIG. 1 illustrates a total hemoglobin index system including a total hemoglobin (tHb) index monitor and a corresponding disposable tHb index optical sensor responsive to pulsatile blood flow within a fingertip;

FIG. 2 is a general flow diagram of tHb index monitoring utilizing a combination of optical sensors including an index sensor and a high-resolution sensor;

FIGS. 3A-B are a tHb index sensor characterization flow diagram and a corresponding sensor characterization graph;

FIGS. 4A-B are a tHb high-resolution sensor characterization flow diagram and corresponding sensor characterization graph;

FIG. 5 is a detailed flow diagram of tHb testing utilizing a combination of index and high-resolution disposable optical sensors;

FIG. 6 is a handheld-monitor tHb index display;

FIG. 7 is a multi-parameter monitor display illustrating a tHb index in conjunction with various other pulsatile blood flow parameters;

FIGS. 8A-B are perspective and top views, respectively, of a tHb index sensor;

FIGS. 9A-E are top and bottom head tape assembly views, enlarged detector window and emitter window views and a connector schematic view, respectively, of a tHb index sensor.

FIG. 10 is an exploded view of a tHb index sensor;

FIGS. 11A-D are top, top-perspective, side and bottom-perspective views, respectively, of a tHb sensor emitter assembly;

FIGS. 12A-D are top, top-perspective, side and bottom-perspective views, respectively, of a tHb sensor detector assembly;

FIGS. 13A-B are LED layout and corresponding emitter schematic views of an emitter assembly; and

FIGS. 14A-B are a socket and plug perspective view and a socket and plug perspective cutaway view of a pogo pin connector assembly.

### DETAILED DESCRIPTION OF THE PREFERRED EMBODIMENTS

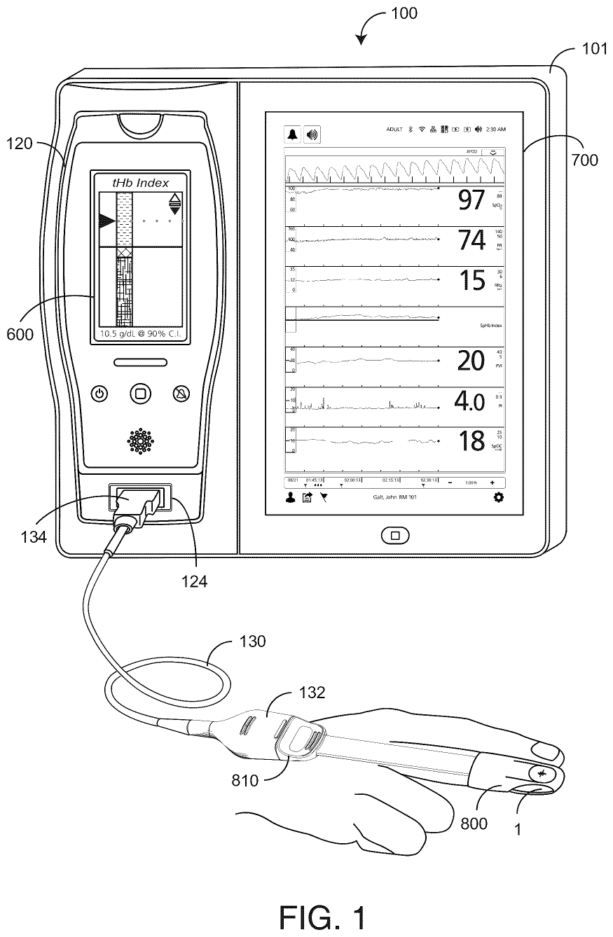

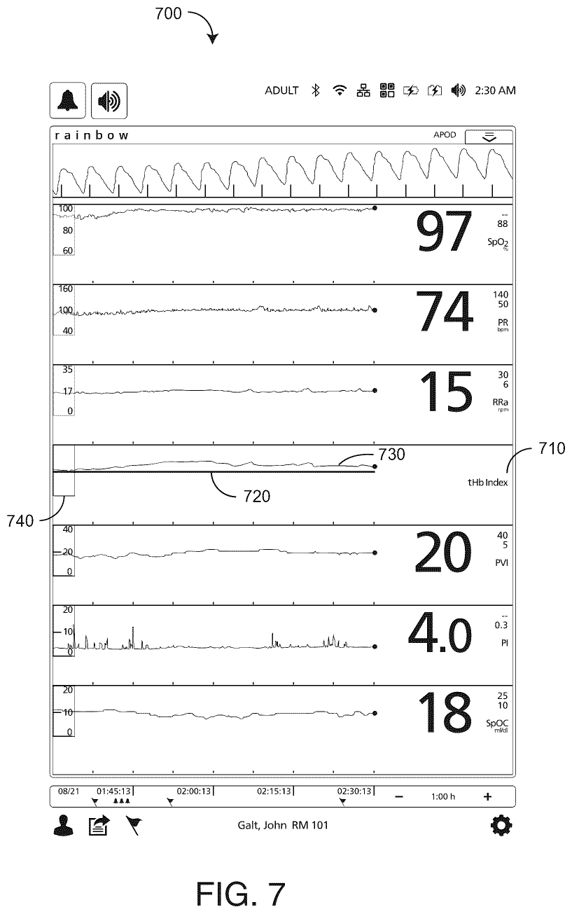

FIG. 1 illustrates a total hemoglobin (tHb) index system 100 including a standalone monitor 101, a portable handheld monitor 120 and a disposable optical sensor 800. The standalone monitor 101 has a main display 700 and the removable handheld monitor 120 has a handheld display 600. The optical sensor 800 attaches to a fingertip 1 so as to optically analyze blood constituents therein, including tHb and others described with respect to FIG. 7, below. In a particular embodiment, the sensor 800 has emitters (LEDs) capable of irradiating the tissue site 1 with multiple wavelengths of light and corresponding detectors capable of detecting the light after attenuation by pulsatile blood flow within the tissue site 1. The sensor 800 removably connects to a patient cable 130, which connects to the monitor 101. In particular, the patient cable 130 has a sensor-side connector 132 that removably attaches to and electrically communicates with the sensor connector 810 and a monitor-side connector 134 that removably attaches to and electrically communicates with the handheld monitor via a handheld connector 124. The handheld monitor 120 has a monitor mount connector (not visible) that allows the sensor 800 to communicate with the monitor 101. Blood parameter measurements responsive to the index sensor 800 are displayed on the handheld display 600 and/or the larger monitor display 700. A patient monitoring platform responsive to optical sensors among others and providing both handheld and standalone monitoring capabilities is available from Masimo Corporation, Irvine, CA.

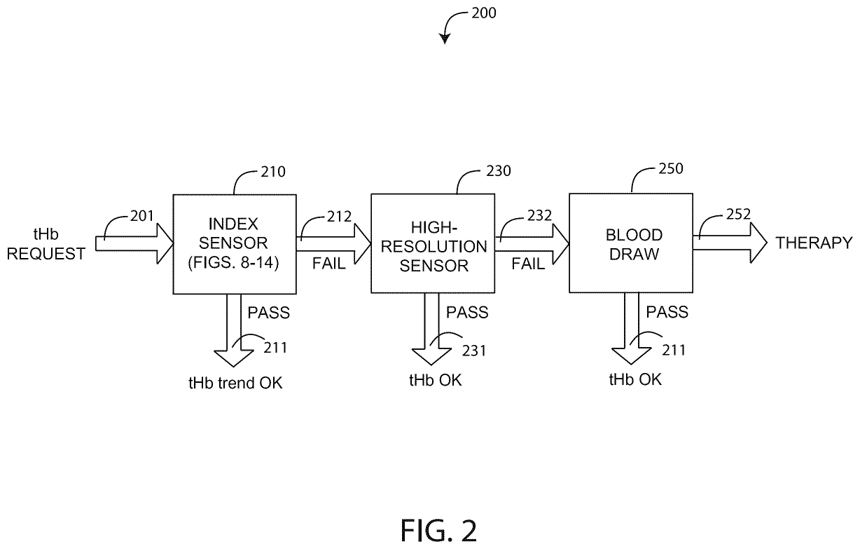

As shown in FIG. 1, an optical sensor 800 disposed of after a single patient use is relatively expensive. Reducing the number of sensor emitters may significantly reduce the cost of testing patients for some conditions, such as blood-loss. In an advantageous embodiment, at least two sensors are provided for medical use. A relatively inexpensive “index sensor” has a reduced set of emitters (fewer wavelengths) and measures a blood parameter, such as tHb, with a correspondingly lower resolution. A relatively expensive high resolution or “hi-res” sensor has an expanded set of emitters (more wavelengths) and measures blood parameters with greater accuracy. In an advantageous embodiment, described in further detail with respect to FIGS. 2-14 below, a total hemoglobin index system derives a unit-less tHb index that varies over time. A tHb index advantageously utilizes a less expensive index sensor with a minimal number of LED emitters so as to reduce disposable sensor costs during initial patient testing.

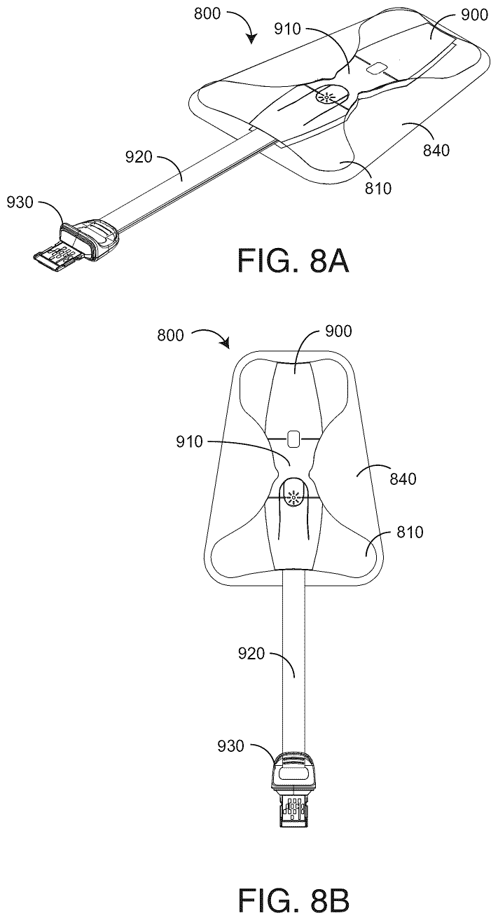

FIG. 2 illustrates total hemoglobin (tHb) index monitoring 200 that advantageously uses a fewer-LED (index) sensor 210 for an initial tHb test. A high resolution (hi-res) sensor 230 that uses more-LEDs is used only if tHb issues, such as potential internal bleeding, cannot be eliminated by the index sensor 210. Both the index sensor 210 and the hi-res sensor 230 are single-use, disposable sensors. The hi-res sensor 230 is substantially more expensive than the index sensor 250 due to LED costs. In a particular embodiment, the index sensor 210 uses four LEDs and the high-res sensor uses eight LEDs. A tHb index sensor embodiment is described in detail with respect to FIGS. 8-14, below. A hi-res sensor is described with respect to U.S. patent application Ser. No. 12/056,179 filed Mar. 26, 2008, titled Multiple Wavelength Optical Sensor, assigned to Masimo Corporation, Irvine, CA and incorporated in its entirety by reference herein.

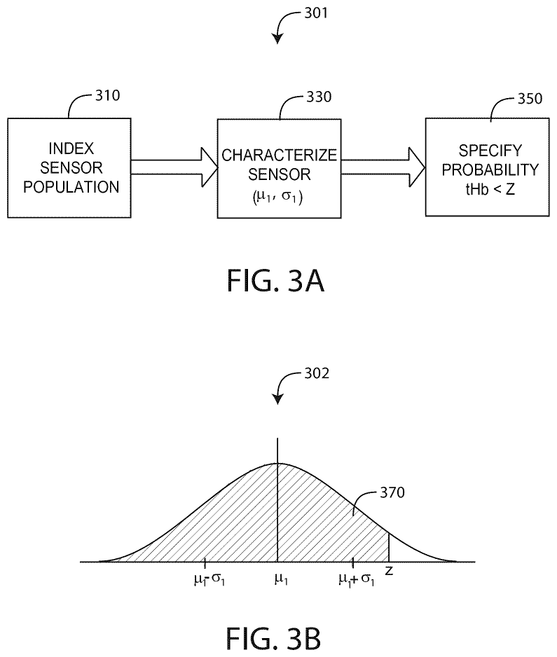

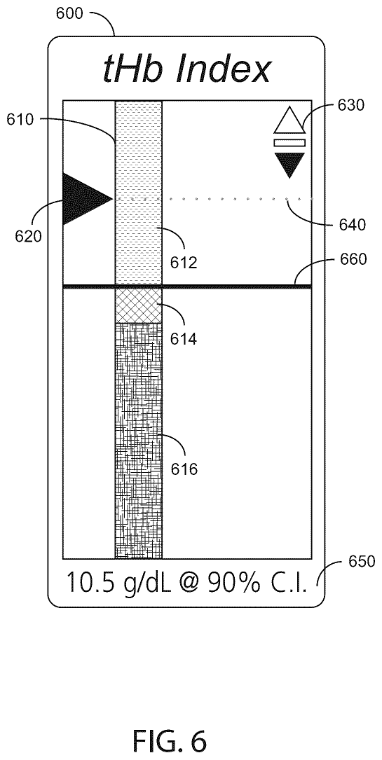

As shown in FIG. 2, a total hemoglobin (tHb) index 200 embodiment begins with a tHb measurement request 201 initially fulfilled with a tHb index sensor 210. The relatively low cost, low resolution index sensor is advantageously utilized to derive a dimensionless trend 211. For example, a stable trend over a sufficiently long period of time may be sufficient assurance to a care provider that further testing is unnecessary, as described with respect to FIG. 3, below. Dimensionless tHb trend displays are described with respect to FIGS. 6-7, below.

Also shown in FIG. 2, if a tHb trend is inconclusive 212, a care provider may request a high-resolution sensor 230. If noninvasive measures fail to yield a definitive diagnosis, an invasive blood draw 260 and corresponding laboratory analysis is utilized alone or in addition to continuous high resolution sensor measurements for a definitive tHb assessment. Appropriate patient therapy 252 may include follow-up surgery and blood transfusion.

FIGS. 3A-B illustrate a tHb index sensor method including a characterization flow diagram 301 and a corresponding sensor characterization graph 302. As shown in FIG. 3A, a statistically-significant population of index tHb sensors 310 is characterized 330 by measuring the mean (μ1) and standard deviation (σ₁) of a Gaussian-distributed population of tHb measurements. The corresponding z score of the index sensors 350 is calculated as:

$$z = \frac{x - {μ}_{1}}{{σ}_{1}} \qquad EQ . 1$$

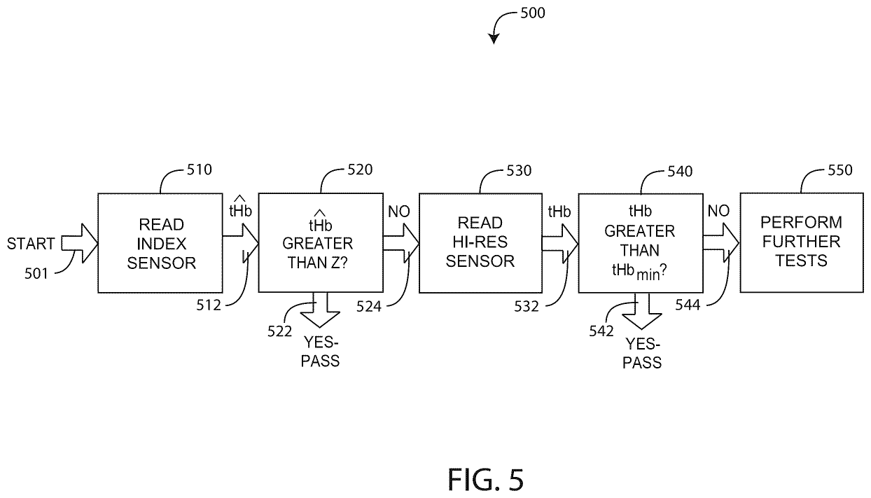

As shown in FIG. 3B, the probability that a tHb measurement for a given index sensor is less than z is the shaded area 370 of a normal distribution having a mean μ₁ and a standard deviation σ₁. For example, from a standard statistical table, a z value of 1.28 indicates a 89.97% (˜90%) probability that the measured value is less than z. Use of a index sensor z value for initial tHb index 210 (FIG. 2) is described in further detail with respect to FIG. 5, below.

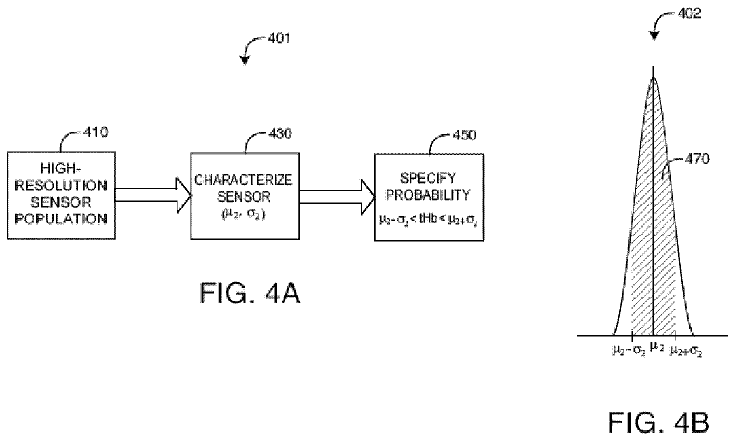

FIGS. 4A-B illustrate a high-resolution tHb sensor characterization method including a characterization flow diagram 401 and a corresponding sensor characterization graph 402. As shown in FIG. 4A, a statistically-significant population of high-resolution tHb sensors 410 is characterized 430 by measuring the mean (μ₂) and standard deviation (σ₂) of a Gaussian-distributed population of tHb measurements. The corresponding accuracy of the high-resolution sensor is calculated as:

$$tHb = {μ}_{2} \pm {σ}_{2} \qquad EQ . 2$$

As shown in FIG. 4B, the probability that a tHb measurement for a given high-resolution sensor is within one standard deviation of the mean is 68%. Use of a high-resolution sensor tHb measurement is described in further detail with respect to FIG. 5, below.

FIG. 5 further illustrates tHb testing 500 utilizing a combination of a disposable index sensor and a disposable high-resolution sensor. Initially, a disposable tHb index sensor is attached to a patient and a corresponding monitor 101 (FIG. 1). The monitor reads the index sensor 510 to obtain a total hemoglobin estimate [symbol] . That estimate is compared with the z-value 520, as described with respect to EQ. 1, above. If the total hemoglobin estimate is greater than z, then the patient has a sufficiently high tHb to pass the test 522, i.e. to eliminate low tHb and issues regarding low tHb (such as internal bleeding) as a concern. In an embodiment, z is chosen so that the probability that tHb is too low is approximately 90%. Advantageously, use of a relatively inexpensive, minimally emitter-populated index sensor at this stage of patient testing provides a significant cost-saving over time and encourages more frequent use of noninvasive tHb testing as a patient care standard.

Further shown in FIG. 5, in the event [symbol] is less than z 524 then tHb is retested utilizing a disposable high resolution sensor 530. If tHb is greater than tHbₘᵢₙ, then the patient has a sufficiently high tHb to pass the test 542. Advantageously, even though a second, more expensive fully emitter-populated high-resolution sensor is used during this second stage test, this multistage test is configured to have an overall cost savings as compared to solely using high-resolution sensors. Although an index sensor test described above is based upon a z-value, other tHb threshold measures may be used. As an example, a fraction of or multiplier of z may be used to calculate the index sensor threshold.

Traditional invasive lab testing provides delayed results and requires a painful needle stick and time-consuming blood draws. A total hemoglobin (tHb) index system incorporating an advantageous noninvasive, disposable sensor and a monitor advantageously calculating and displaying a tHb index facilitates timely patient assessment and reduces the need to wait for lab results.

Also, noninvasive tHb monitoring advantageously provides real-time visibility to changes, or lack of changes, in total hemoglobin between invasive blood sampling. Continuous, real-time tHb monitoring is particularly advantageous when a tHb trend is stable and a clinician may otherwise think tHb is dropping; a tHb trend is rising and the clinician may otherwise think tHb is not rising fast enough; or the tHb trend is dropping and the clinician may otherwise think tHb is stable.

FIGS. 6-7 illustrate advantageous graphical index measures of total hemoglobin (tHb index) that address the need to provide real-time visibility to changes while using a relatively inexpensive tHb index sensor. FIG. 6 illustrates a tHb index display 600 embodiment advantageously suited for a relatively small area display, such as on a handheld monitor 120 (FIG. 1). In particular, the handheld tHb index has no time axis. Instead, a vertical bar graph 610 has dimensionless green 612, yellow 614 and red 616 zones for indicating a range of relative tHb values. The present relative value of a tHb index is indicated by a vertical-ranging pointer 620 and a dotted line 640 extending horizontally across the display 600. A horizontal line 660 is disposed across the width of the display between the green zone 612 and yellow zone 614. A tHb value 650 is listed at the display bottom, e.g. “10.5 g/dL @ 90% C.I.” Accordingly, the horizontal line 660 represents 10.5 g/dl and a corresponding 90% confidence interval that the present tHb value is greater than 10.5 g/dL. In this example, the yellow zone 614 represents a 10% likelihood the present tHb value is less than 10.5 g/dL. Although the tHb index display has no time axis, a trend indicator 630 has up arrow, horizontal line and down arrow symbols. The current tHb index trend is indicated by a highlighted one of these symbols.

FIG. 7 illustrates a multi-parameter monitor display 700 including such parameters as oxygen saturation, pulse rate, respiration rate, tHb index 710, pulse variability index (PVI), perfusion index (PI) and oxygen content (SpOC) versus time. An advantageous tHb index versus time display 710 has no listed parameter range 740. However, a baseline tHb value 720 is displayed as a horizontal line and a time varying tHb index 730 is displayed relative to the baseline 720. The baseline 720 is set at a tHb value and corresponding confidence interval, as described with respect to FIG. 6, above.

FIGS. 8A-B generally illustrate a disposable index sensor 800 that removably attaches to a fingertip and electrically interconnects to a physiological monitor as shown and described with respect to FIG. 1, above. The sensor 800 has an adhesive butterfly wrap 810, a head assembly 900 and a release liner 840. The head assembly 900 has a sensor head 910, a connector 930 and an insulated interconnect 920. The interconnect 920 provides mechanical and electrical communications between the sensor head 910 and the connector 930. The release liner 840 is removed from the adhesive butterfly wrap 810 so as to attach the sensor head 910 to a fingertip 1 (FIG. 1). A connector 930 inserts into a corresponding patient cable socket 132 (FIG. 1) so as to provide communications between the sensor 800 and a standalone monitor 101 (FIG. 1) or portable handheld monitor 120 (FIG. 1).

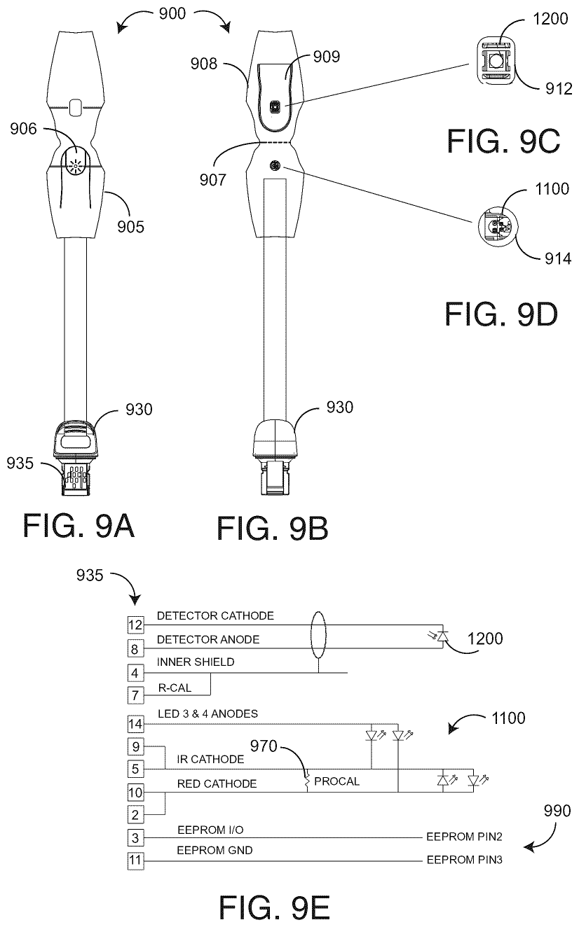

FIGS. 9A-E further illustrate the head tape assembly 900 including a first head tape side (FIG. 9A), a second head tape side (FIG. 9B), a detector window 912 (FIG. 9C) exposing the detector 1200, an emitter window 914 (FIG. 9D) exposing the emitter 1100 and a connector 935 (FIG. 9E) schematic view, respectively, for a tHb index sensor. As shown in FIG. 9A, the first head tape side has an imprinted fingernail target 906 on a bottom half 905. As shown in FIG. 9B, the second head tape side has an imprinted finger pad target 909 on a top half 908 and a printed fold line 907 separating the top half 908 and bottom half 905. The sensor head 910 (FIGS. 8A-B) is attached to a fingertip site by placing the fingernail target 906 over a fingernail, folding the sensor head at the fold line 907 and over the fingertip so as to place the finger pad target on a finger pad. The sensor head 910 is then held in place by folding the butterfly wrap 810 (FIGS. 8A-B) around the finger 800 (FIG. 1).

As shown in FIG. 9E, the connector 930 (FIG. 9A) and in particular, the connector contacts 935 allow a monitor to sequentially activate the LEDs 1100, which illuminate a fingertip with red and IR wavelengths. The detector 1200 is responsive to the wavelengths after attenuation by pulsatile blood flow within the fingertip. The monitor analyzes the detector signal so as to measure blood constituents including tHb. The monitor may also read an EEPROM 990 and resistor 970 mounted in the connector 930 so as to identify the sensor 800 (FIGS. 8A-B).

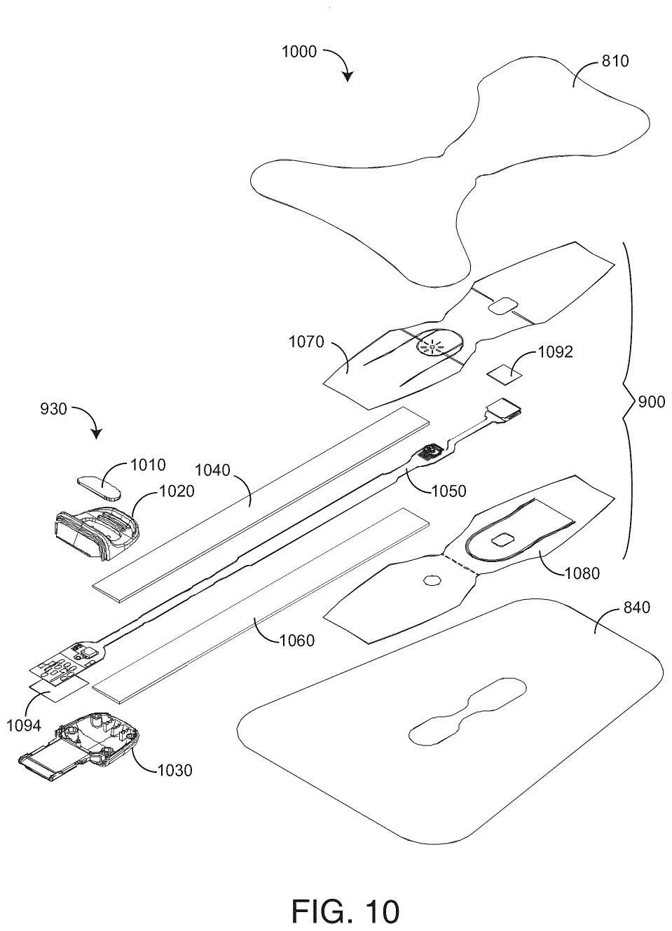

FIG. 10 further illustrates the tHb index sensor described above with respect to FIGS. 8-9. The tHb index sensor 1000 (800 FIGS. 8A-B) has an adhesive butterfly wrap 810, a top head tape 1070, a polyethylene foam tape insulator 1040, 1060, a flex circuit assembly 1050, a bottom head tape 1080 and a release liner 840. The sensor connector 930 has an LED label 1010 identifying this as a index sensor, a connector top shell 1020, a sensor bottom shell 1030 and adhesives 1092, 1094.

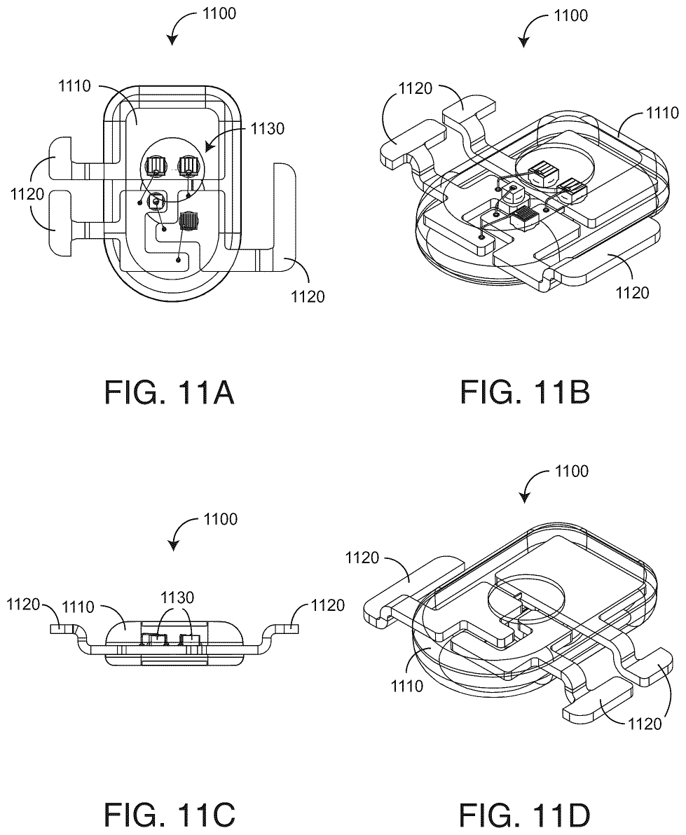

FIGS. 11-13 illustrate in detail the optical elements of a tHb index sensor 800 (FIGS. 8A-B), including an emitter 1100 that sequentially illuminates a fleshy tissue site, such as a fingertip, with four discrete wavelengths of optical radiation and a detector 1200 that is responsive to the optical radiation after absorption by pulsatile blood flow within the tissue site. A monitor 100 (FIG. 1) has processors responsive to the detector 1200 so as to derive an indication of one or more blood constituents, such as a tHb trend described with respect to FIGS. 6-7 above.

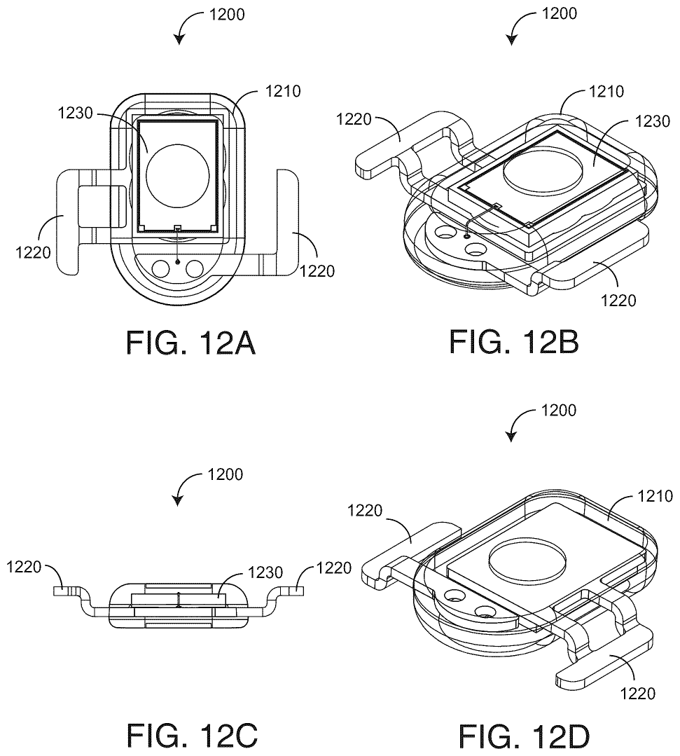

As shown in FIGS. 11A-D, an emitter 1100 has an encapsulant 1110 housing a lead frame 1120 and emitter dice 1130 mounted and wire bonded to the lead frame 1120. As shown in FIGS. 12A-D, a detector 1200 has an encapsulant 1210 housing a lead frame 1220 and a detector die 1230 mounted and wire bonded to the lead frame 1220. The emitter 1100 and detector 1200 are mounted via their respective lead frames 1120, 1220 to a flex circuit assembly 1050 (FIG. 10), as described above, so as to respond to emitter drive signals from a monitor via a connector 935 (FIG. 9E) and to transmit detector signals to a monitor via the connector 935 (FIG. 9E).

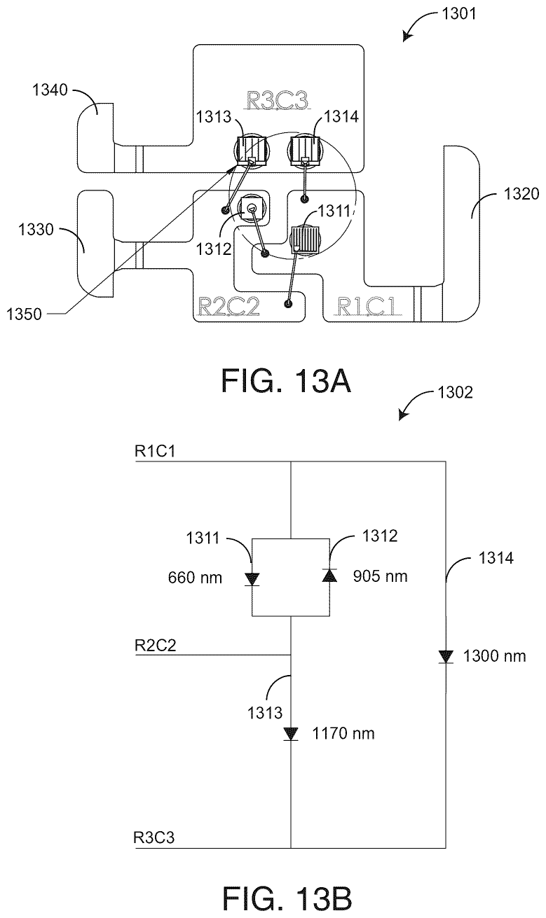

As shown in FIGS. 13A-B, in a 4-wavelength embodiment, LEDs are mounted on a lead frame 1301 having three leads 1320, 1330, 1340. Four LED die 1311-1314 are each mounted on one face and wire bonded on an opposite face to various lead frame pads RIC1, R2C2, R3C3 proximate an emitter optical center 1350. In a particular embodiment, the LEDs emit light at 660 nm, 905 nm, 1170 nm and 1300 nm.

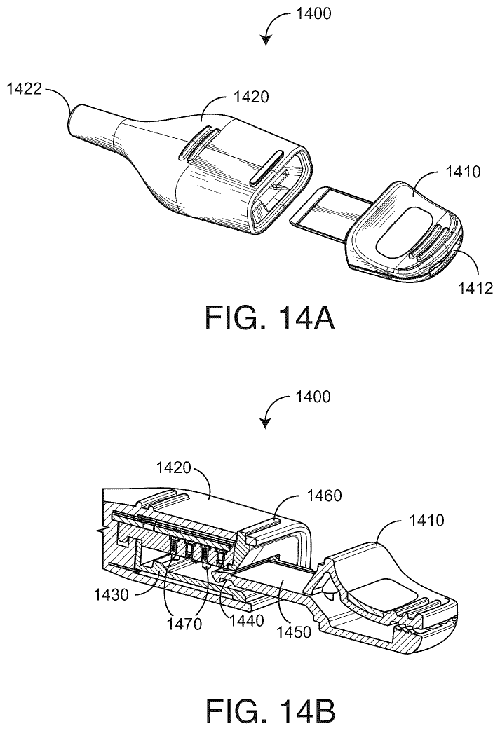

FIGS. 14A-B illustrate a pogo pin sensor connector assembly. As shown in FIG. 14A, the connector assembly 1400 has a plug 1410 that accepts flex circuit conductors via a generally elongated aperture 1412 and a socket 1420 that accepts cable conductors via a generally round aperture 1422. A plug shelf 1450 provides a generally solid, flat surface for fixedly mounting flex circuit connector pads e.g. 935 (FIG. 9A), which are inserted into the socket 1420. The plug 1410 provides a sensor connector e.g. 810 (FIG. 1) and the socket 1420 provides a monitor cable connector 132 (FIG. 1) so as to allow a monitor and sensor to electrically communicate drive signals and sensor signals as described above.

As shown in FIG. 14B, when the plug 1410 is fully inserted into the socket 1420, a plug latch 1440 engages a socket catch 1430 removably securing the plug 1410 to the socket 1420. Spring-mounted pogo pins 1470 in the socket 1420 mechanically and electrically engage flex circuit pads on the plug 1410 so as to electrically interconnect flex circuit and cable conductors. A release 1460 disengages the catch 1430 and latch 1440 allowing the plug 1410 to be removed from the socket 1420.

A total hemoglobin index system has been disclosed in detail in connection with various embodiments. These embodiments are disclosed by way of examples only and are not to be construed as limiting the scope of the claims that follow. One of ordinary skill in art will appreciate many variations and modifications.

1. (canceled)

2. A physiological monitoring system for measuring total hemoglobin in a wearer, the physiological monitoring system comprising:
a first sensor configured to measure a first total hemoglobin estimate with a first resolution, the first sensor comprising:
a first plurality of LEDs and a first detector, the first plurality of LEDs configured to emit light at one or more wavelengths at a measurement site of a wearer, the first detector configured to generate a first physiological signal associated with the one or more light wavelengths after attenuation by tissue at the measurement site; and
one or more hardware processors in communication with the first sensor and a display, the one or more hardware processors configured to:
receive the first physiological signal from the first detector;
determine the first total hemoglobin estimate based at least in part on the first physiological signal;
determine whether the first total hemoglobin estimate is below a predetermined threshold; and
responsive to a determination that the first total hemoglobin estimate is below the predetermined threshold, cause presentation of a visual indication in a graphical user interface of the display for visualization by the wearer such that the wearer is informed of a need to use a second sensor for measurement of a second total hemoglobin estimate,
wherein the second sensor is configured to measure the second total hemoglobin estimate with a second resolution that is greater than the first resolution, the second sensor comprising:
a second plurality of LEDs and a second detector, the second plurality of LEDs configured to emit light at one or more wavelengths at the measurement site of the wearer, the second detector configured to generate a second physiological signal associated with the one or more light wavelengths after attenuation by tissue at the measurement site.

3. The physiological monitoring system of claim 2, wherein the second plurality of LEDs comprises more LEDs than does the first plurality of LEDs.

4. The physiological monitoring system of claim 2, wherein the one or more hardware processors are further configured to determine one or more of at least oxygen saturation, respiration rate, pulse rate, and pulse rate variability of the wearer based at least in part on the first physiological signal.

5. The physiological monitoring system of claim 2, wherein the one or more hardware processors are further configured to determine a total hemoglobin trend of the wearer based at least in part on the first physiological signal over time.

6. The physiological monitoring system of claim 2, wherein the visual indication is configured to dynamically change responsive to a change in a total hemoglobin estimate of the wearer.

7. The physiological monitoring system of claim 2, wherein the one or more hardware processors are configured to continuously measure physiological data by sampling the first sensor at a sampling rate configured to capture real-time changes in total hemoglobin of the wearer.

8. The physiological monitoring system of claim 2, wherein the physiological monitoring system further comprises the display.

9. The physiological monitoring system of claim 2, wherein the measurement site comprises pulsatile blood flow.

10. The physiological monitoring system of claim 2, wherein the first plurality of LEDs comprises a plurality of LED channels.

11. The physiological monitoring system of claim 10, wherein the first sensor is further configured to sequentially emit light at different wavelengths at the measurement site of the wearer.

12. A method for measuring total hemoglobin in a wearer, the method comprising:
under control of one or more hardware processors, the one or more hardware processors in communication with a display and a first sensor configured to measure a first total hemoglobin estimate with a first resolution, the first sensor comprising a first plurality of LEDs configured to emit light at one or more wavelengths at a measurement site of the wearer and a first detector configured to generate a first physiological signal associated with the one or more light wavelengths after attenuation by tissue at the measurement site:
receiving the first physiological signal from the first detector;
determining the first total hemoglobin estimate based at least in part on the first physiological signal;
determining whether the first total hemoglobin estimate is below a predetermined threshold; and
responsive to determining that the first total hemoglobin estimate is below the predetermined threshold, causing presentation of a visual indication in a graphical user interface of the display for visualization by the wearer such that the wearer is informed of a need to use a second sensor for measurement of a second total hemoglobin estimate,
wherein the second sensor is configured to measure the second total hemoglobin estimate with a second resolution that is greater than the first resolution, the second sensor comprising:
a second plurality of LEDs and a second detector, the second plurality of LEDs configured to emit light at one or more wavelengths at the measurement site of the wearer, the second detector configured to generate a second physiological signal associated with the one or more light wavelengths after attenuation by tissue at the measurement site.

13. The method of claim 12, wherein the second plurality of LEDs comprises more LEDs than does the first plurality of LEDs.

14. The method of claim 12, the method further comprising, under control of the one or more hardware processors:
determining one or more of at least oxygen saturation, respiration rate, pulse rate, and pulse rate variability of the wearer based at least in part on the first physiological signal.

15. The method of claim 12, the method further comprising, under control of the one or more hardware processors:
determining a total hemoglobin trend of the wearer based at least in part on the first physiological signal over time.

12. The method of claim 12, wherein the visual indication is configured to dynamically change responsive to a change in a total hemoglobin estimate of the wearer.

17. The method of claim 12, method further comprising, under control of the one or more hardware processors:
continuously measuring physiological data by sampling the first sensor at a sampling rate configured to capture real-time changes in total hemoglobin of the wearer.

18. The method of claim 12, the method further comprising providing the display.

19. The method of claim 12, wherein the measurement site comprises pulsatile blood flow.

20. The method of claim 12, wherein the first plurality of LEDs comprises a plurality of LED channels.

21. The method of claim 20, wherein the first sensor is further configured to sequentially emit light at different wavelengths at the measurement site of the wearer.
